(19) United States
(12) Patent Application Publication
(10) Pub. No.: US 2012/0258546 A1
(43) Pub. Date: Oct. 11, 2012

(54) Automated On-Instrument pH Adjustment

(75) Inventors: David MARRAN, Durham, CT (US); Mark BEAUCHEMIN, S Glastonbury, CT (US)

(73) Assignee: LIFE TECHNOLOGIES CORPORATION, Carlsbad, CA (US)

(21) Appl. No.: 13/441,565

(22) Filed: Apr. 6, 2012

Related U.S. Application Data
(60) Provisional application No. 61/473,402, filed on Apr. 8, 2011.

(51) Int. Cl. G01N 33/84

(52) U.S. Cl. 436/163

(57) ABSTRACT

A method of preparing a sequencing device includes determining a sensitivity of a pH of a solution to a first reagent, determining an amount of the first reagent to add to the solution to approach a target pH, adding the amount of the first reagent to the solution, and diluting a nucleotide solution with the solution.

### CROSS-REFERENCE TO RELATED APPLICATION(S)

This application claims benefit of U.S. Provisional Application No. 61/473,402, filed Apr. 8, 2011 and entitled “AUTOMATED ON-INSTRUMENT pH ADJUSTMENT,” which is incorporated herein by reference in its entirety.

### FIELD OF THE DISCLOSURE

This disclosure, in general, relates to methods for preparing a sequencing device and sequencing devices.

### BACKGROUND

Instrument platforms for label-free/non-optical nucleic acid sequencing have recently become available. For example, the Personal Genome Machine (PGM™) developed Ion Torrent Systems Inc. uses a specialized flowcell with integrated semiconductor sensors for nucleic acid sequencing. This system is able to perform nucleic acid sequence analysis by ion detection-based methods with high precision and sensitivity. In one exemplary approach, microwells containing template nucleic acid strands to be sequenced are exposed to nucleotide solutions of selected types (e.g. G, A, T, C). Under the appropriate conditions, when the introduced nucleotide is complementary to a leading template nucleotide, an enzyme (e.g., polymerase) incorporates the nucleotide into a growing complementary strand. The nucleotide incorporation results in the release of hydrogen ions that are detected by ion sensors, thus providing a mechanism to detect the incorporation events directly. Further details of this instrumental approach are described in Rothberg et al, U.S. patent publication 2009/0127589 and Rothberg et al, U.K. patent application GB24611127.

Such systems can detect localized changes in solution pH at each sensor when nucleotide incorporation takes place. Variations in pH of reagents can cause detection errors.

### SUMMARY

The present teachings are directed to apparatus, methods, and software for automated measurement or adjustment of reagent and solution pH in a sequencing system. Such approaches may implement automated or semi-automated protocols which reduce user intervention while desirably improving the accuracy and timeliness of pH adjustments. Such approaches may also be configured to operate in a monitoring mode so as to be able to correct for pH changes that may occur over time.

These above-characterized aspects, as well as other aspects, of the present teachings are exemplified in a number of illustrated implementation and applications, some of which are shown in the figures and characterized in the claims section that follows. However, the above summary is not intended to describe each illustrated embodiment or every implementation of the present teachings.

### BRIEF DESCRIPTION OF THE DRAWINGS

The present disclosure may be better understood, and its numerous features and advantages made apparent to those skilled in the art by referencing the accompanying drawings.

FIG. 1 illustrates an exemplary flow cell and sensor array according to the present teachings.

FIG. 2 illustrates an exemplary cross-section of a flow cell and sensor according to the present teachings.

FIG. 3 illustrates an exemplary nucleic acid sequencing method according to the present teachings.

FIG. 4 illustrates an exemplary workflow for preparing a flow cell and sensor array for runtime operation according to the present teachings.

FIG. 5 illustrates an exemplary method for pH adjustment of solutions according to the present teachings.

FIG. 6 illustrates an exemplary pH measurement and adjustment procedure according to the present teachings.

FIG. 7 illustrates an exemplary report of a pH procedure according to the present teachings.

FIG. 8 illustrates an exemplary graphical runtime analysis of pH determination according to the present teachings.

FIG. 9 illustrates an exemplary comparison of signal response between manual pH adjustment and pH adjustment methods according to the present teachings.

FIG. 10 illustrates an exemplary method for monitoring error status according to the present teachings.

FIG. 11 illustrates an exemplary output of pH measurements used for reagent monitoring according to the present teachings.

FIG. 12 illustrates an exemplary method for pH adjustment.

The use of the same reference symbols in different drawings indicates similar or identical items.

### DETAILED DESCRIPTION

While the present teachings are amenable to various modifications and alternative forms, specifics thereof have been shown by way of example in the drawings and will be described in detail. It should be understood, however, that the intention is not to limit the present teachings to the particular embodiments described. On the contrary, the intention is to cover all modifications, equivalents, and alternatives falling within the scope of the present teaching.

Particular sequencing systems detect localized changes in solution pH at each sensor when nucleotide incorporation takes place. Such sequencing systems are particularly sensitive to variations in pH of reagents. In particular, variations in reagent pH can lead to detection errors. For detection to occur, the pH of the reagents and solutions used in connection with the instrument should be within certain desired ranges. To this end, current methods for instrument and reagent preparation utilize careful measurement and adjustment of pH prior to conducting a sequencing run. Such methods can be laborious, time-consuming, and require the use of expensive secondary instrumentation (e.g. highly sensitive pH meters). Moreover, Applicants have discovered that such techniques introduce errors and are unreliable.

Variability in pH may be introduced as a result of differences in user technique, pH meter calibration, reagent quality, water quality, presence of contaminants and the like. In particular, Applicants have discovered that reagents that have been previously calibrated can be subject to undesirable pH shifts, for example, as a result of dissolved atmospheric carbon dioxide in the reagent solutions which may have the effect of gradual lowering the pH over time. Further, the particular type of material which is used to contain or store various reagents may directly affect the solution pH contained therein as a result of materials leaching into the solution from the containment vessel itself or because of gas-permeable characteristics that allow atmospheric gas diffusion to take place, changing the pH of solutions contain therein.

In the case of nucleic acid sequencing, pH drift from an initial measured pH to different pHs over time (for example from pH 7.5 to pH 6.8) can affect overall system accuracy or increase signal noise, which can decrease overall sensitivity of the system during a run. The present technique provides improved control over the pH of solutions and improved sequencing performance in sequencing systems.

In one aspect, the present teachings relate to apparatuses and methods for carrying out and monitoring a plurality of multi-step reactions with electronic sensors. The multi-step reactions may be cyclic, such as in polynucleotide sequencing reactions, polynucleotide synthesis reactions, or the like, where repeated cycles of one or more steps are carried out, or they may be non-cyclic, such as in multi-component labeling reactions, as for example, in a sandwich assay using enzymatic labels. Multi-step reactions may also result from the presence of a biological material, such as living cells or tissue sample, where responses, e.g. the presence or absence of metabolites, are detected in response to a series of reagent exposures, which may be drug candidate molecules, or the like. Electronic sensors of the present teachings may be integrated into a sensor array suitable for sensing individual reactions taking place on or adjacent to a surface of the array. In one embodiment, an array of reaction confinement regions is integral with such a sensor array. An array of reaction confinement regions may take the form of a microwell array or a reaction chamber array made by conventional micro- or nanofabrication techniques, for example, as described in Rothberg et al, U.S. patent publication US2009/0127589 and Rothberg et al, U.K. patent application GB24611127.

In one embodiment, each microwell or reaction chamber in such an array has at least one sensor that is in a sensing relationship so that one or more characteristics of a reaction in the microwell or reaction chamber can be detected or measured. Typically, such electronic sensors measure directly or indirectly (for example, by the use of a binding compound or label) reaction byproducts including, but not limited to, chemical species resulting from a reaction, such as an increase or decrease in pH, or physical changes caused by a reaction, such as increases or decreases in temperature, e.g. as disclosed in Rothberg et al (U.S. and U.K. patent publications cited above). Electronic sensors of the present teaching may convert changes in the presence, concentration or amounts of reaction byproducts into an output signal, which may be a change in a voltage level or a current level which, in turn, may be processed to extract information about a reaction. Electronic sensors of the array, or a subset of such sensors, may also be used to monitor the presence or concentration of reactants, indicator molecules, or other reagents, such as reagents for identifying microwells containing analytes.

The structure or design of sensors for use with the present teachings may vary widely, as exemplified by the following, which are incorporated by reference: Rothberg et al, U.S. patent publication US2009/0127589; Rothberg et al, U.K. patent application GB24611127; or the like. In a selected embodiment, sensors of the array comprise at least one chemically sensitive field effect transistor configured to generate at least one output signal related to a property of a chemical reaction in proximity thereof. In particular, the sensors can include an array of chemically sensitive field effect transistors (chemFET). Such properties may include a concentration (or a change in concentration) of a reactant or product, or a value of physical property (or a change in such value), such as temperature.

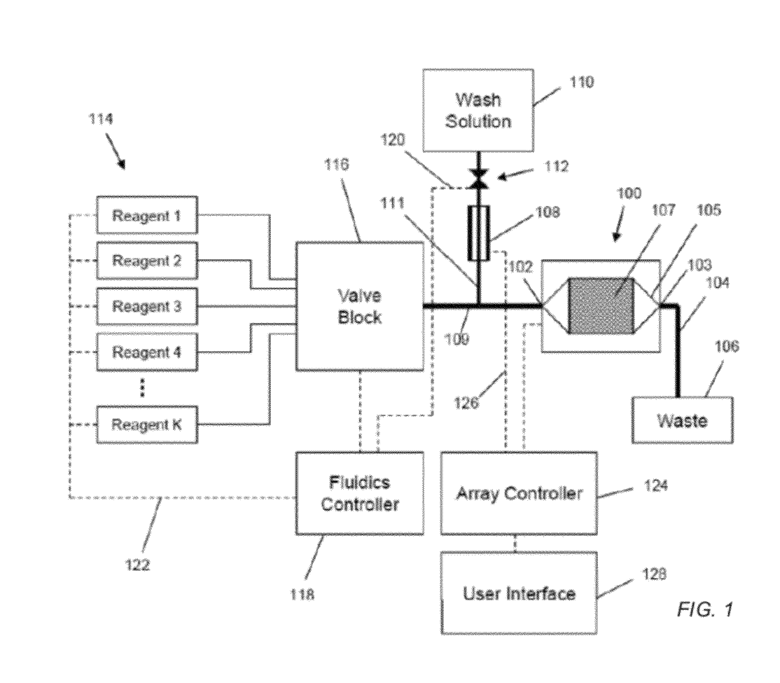

Components of one embodiment are illustrated diagrammatically in FIG. 1. A flow cell and sensor array 100 comprises an array of reaction confinement regions (which may comprise a microwell array) that is operationally associated with a sensor array, so that, for example, each microwell has a sensor suitable for detecting an analyte or reaction property of interest. In various embodiments, a microwell array may be integrated with the sensor array on a single substrate or a single chip. A flow cell can have a variety of designs for controlling the path and flow rate of reagents over the microwell array. In some embodiments, a flow cell is a microfluidics device and may be fabricated with micromachining techniques or precision molding to include additional fluidic passages, chambers, and so on. In one aspect, a flow cell comprises an inlet 102, an outlet 103, and a flow chamber 105 for defining the flow path of reagents over the microwell array 107.

Reagents are discarded into a waste container 106 after exiting 104 the flow cell and sensor array 100. A function of the apparatus can be to deliver different reagents to flow cell and sensor array 100 in a predetermined sequence, for predetermined durations, at predetermined flow rates, and to measure physical or chemical parameters in the microwells that provide information about the status of a reaction taking place therein, or in the case of empty wells, information about the physical or chemical environment in the flow cell. To this end, fluidics controller 118 controls by lines 120 and 122 the driving forces for a plurality of reagents 114, wash solutions 110 and the operation of valves (for example, 112 and 116). The reagents and wash solutions may be driven through the fluid pathways, valves and flow cell by pumps, by gas pressure or other conventional methods.

Further components of this embodiment include array controller 124 for providing bias voltages and timing and control signals to the sensor array (if such components are not integrated into the sensor array), and for collecting or processing output signals. Information from flow cell and sensor array 100, as well as instrument settings and controls may be displayed and entered through user interface 128. For some embodiments, for example, nucleic acid sequencing, the temperature of flow cell and sensor array 100 is controlled so that reactions take place and measurements are made at a known, and preferably, a predetermined temperature.

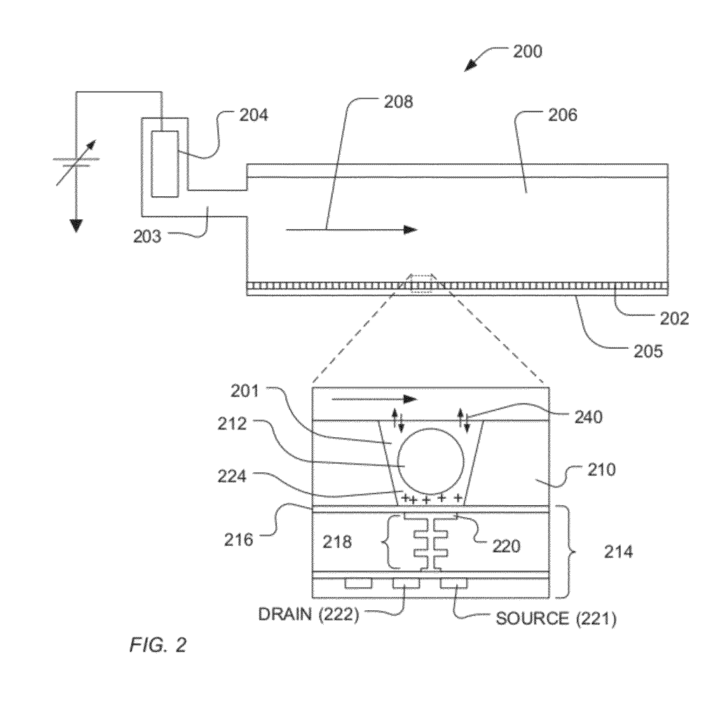

FIG. 2 is an expanded and cross-sectional view of an exemplary flow cell 200 showing a portion 206 of a flow chamber with reagent flow 208 moving across the surface of microwell array 202 over the open ends of the microwells. Microwell array 202 and sensor array 205 together form an integrated unit forming a bottom wall or floor of flow cell 200. In one embodiment, reference electrode 204 is fluidly connected to flow chamber 206. A microwell 201 and sensor 214 are shown in an expanded view. Microwell 201 may be formed by conventional microfabrication technique, as described briefly below. Microwell volume, shape, aspect ratio (such as, base width-to-well depth ratio), and the like, depend on a particular application, including the nature of the reaction taking place, as well as the reagents, byproducts, and labeling techniques (if any) that are employed. Sensor 214 may be configured as a chemical sensitive field effect transistor (chemFET) with floating gate 218 having sensor surface 220 optionally separated from the microwell interior by passivation layer 216. Sensor 214 is predominantly responsive to (and generates an output signal related to) the amount of charge 224 present on the passivation layer 216 opposite of sensor plate 220. Changes in charge 224 cause changes in the current between source 221 and drain 222 of the field effect transistor (FET), which may be used directly to provide a current-based output signal or indirectly with additional circuitry to provide a voltage output signal. Reactants, wash solutions, and other reagents move into microwells from flow chamber 206 by diffusion, convection, or other fluidic principals 240.

Typically reactions carried out in microwells 202 are analytical reactions to identify or determine characteristics or properties of an analyte of interest. Such reactions generate directly or indirectly byproducts that affect the amount of charge adjacent to sensor plate 220. Indirect detection may occur, for example, if byproduct chelators or other binding compounds are used that affect the sensor after binding an analyte of interest, or if labeling moieties are employed, such as enzymes that may generate a secondary byproduct as the result of a binding event, or the like. If such byproducts are produced in small amounts or rapidly decay or react with other constituents, then multiple copies of the same analyte may be analyzed in microwell 201 at the same time in order in increase the output signal ultimately generated. In one embodiment, multiple copies of an analyte may be attached to solid phase support 212, either before or after deposition into a microwell. Solid phase supports 212 may include microparticles, nanoparticles, beads, solid and porous, comprising gels, and the like.

When sensor-active reagent flows into the flow chamber, it moves from flow chamber 206 through microwell 201 that contains particle 212, as well as through other microwells that may or may not contain particles, and to the region of passivation layer 216 opposite of sensor plate 220. In one embodiment, where the sensors are configured to measure pH, a charging reagent may be used as a solution having a predetermined pH, which is used to replace a first reagent at a different predetermined pH. Preferably, the first reagent pH is known and the change of reagents effectively exposes sensors of the microwells to a step-function change in pH, which will produce a rapid change in charge on their respective sensor plates. In one embodiment, a pH change between the first reagent and the charging reagent (or sometimes referred to herein as the “second reagent” or the “sensor-active” reagent) is 2.0 pH units or less; in another embodiment, such change is 1.0 pH unit or less; in another embodiment, such change is 0.5 pH unit or less; in another embodiment, such change is 0.1 pH unit or less. The changes in pH may be made using conventional reagents, e.g. HCl, NaOH, or the like. Exemplary concentrations of such reagents for DNA pH-based sequencing reactions are in the range of from 5 to 200 μM, or from 10 to 100 μM. The variation in charge at a microwell surface opposite a sensor plate indicative of the presence or absence of analyte (or a byproduct from a reaction on an analyte) is measured and registered as a related variation in the output signal of the sensor, e.g. a change in voltage level with time.

As will be described in greater detail hereinbelow, the pH sensitivity of the sensor 220 may also be advantageously used to discern the pH of solutions introduced into the flowcell without a sequencing reaction taking place. Thus, the sensor array also provides the ability to directly monitor the pH of solutions and changes in pH as new solutions are introduced. Such functionality desirably provides a mechanism to conveniently introduce various instrument solutions used for selected experimental protocols and determine beforehand whether these solutions are appropriate for use during runtime operation of the system.

The fluidic, valving and control features of the system provide the ability to selectively introduce one or more of the solutions into the flow cell, measure solution pH, and flush the system as desired to prevent cross-contamination between solutions and errors in pH measurement resulting from carry-over or residual solution present in the flowcell prior to introduction of the next solution to be evaluated. Additionally, as described in greater detail hereinbelow, the system provides the ability to adjust a selected solutions pH by measuring the pH initially, making calculations to determine how to achieve a desired pH, and then directing appropriate pH modifying components into the solution to effectuate the desired pH change via the fluidic, valving, and control features of the system.

In one aspect, dedicated pH modulating reagent reservoirs may be utilized (e.g. acid/base, HCL/NaOH) to dispense pH modifying fluid into a selected solution. However, other existing solutions which are compatible with the selected solution and at a different pH may be used to modify the pH of the selected fluid. Such an approach may be desirable, for example, to reduce the overall number of liquid reservoirs used in the system, reduce the complexity/number of the fluid paths, or to simplify solution or reagent preparation for the user.

In one aspect, the present teachings may be adapted for use with methods and apparatus for carrying out label-free DNA sequencing, and in particular, pH-based DNA sequencing. Briefly, in pH-based DNA sequencing, base incorporations are determined by measuring hydrogen ions that are generated as natural byproducts of polymerase-catalyzed extension reactions. In one embodiment, templates each having a primer and polymerase operably bound are loaded into reaction chambers (such as the microwells disclosed in Rothberg et al, cited above), after which repeated cycles of deoxynucleoside triphosphate (dNTP) addition and washing are carried out. In some embodiments, such templates may be attached as clonal populations to a solid support, such as a microparticle, bead, or the like, and such clonal populations are loaded into reaction chambers.

As used herein, “operably bound” may mean that a primer is annealed to a template so that the primer's 3′ end may be extended by a polymerase and that a polymerase is bound to such primer-template duplex or in close proximity thereof so that binding or extension takes place whenever dNTPs are added. In each addition step of the cycle, the polymerase extends the primer by incorporating added dNTP only if the next base in the template is the complement of the added dNTP. If there is one complementary base, there is one incorporation, if two, there are two incorporations, if three, there are three incorporations, and so on. With each such incorporation there is a hydrogen ion released, and collectively a population of templates releasing hydrogen ions changes the local pH of the reaction chamber.

The production of hydrogen ions is monotonically related to the number of contiguous complementary bases in the template (as well as the total number of template molecules with primer and polymerase that participate in an extension reaction). Thus, when there is a number of contiguous identical complementary bases in the template (i.e., a homopolymer region), the number of hydrogen ions generated, and therefore the magnitude of the local pH change, is proportional to the number of contiguous identical complementary bases. The corresponding output signals are sometimes referred to as “1-mer”, “2-mer”, “3-mer” output signals, and so on. If the next base in the template is not complementary to the added dNTP, then no incorporation occurs and no hydrogen ion is released in which case, the output signal is sometimes referred to as a “0-mer” output signal.

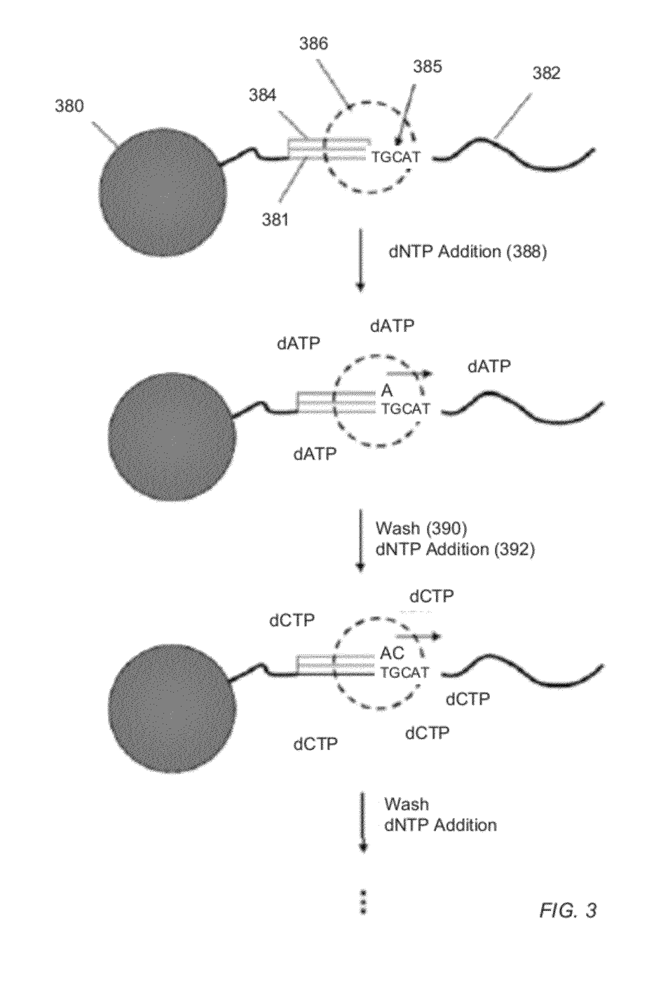

In each wash step of the cycle, an unbuffered wash solution at a predetermined pH is used to remove the dNTP of the previous step in order to prevent misincorporations in later cycles. Usually, the four different kinds of dNTP are added sequentially to the reaction chambers, so that each reaction is exposed to the four different dNTPs one at a time, such as in the following sequence: dATP, dCTP, dGTP, dTTP, dATP, dCTP, dGTP, dTTP, and so on, with each exposure followed by a wash step. The process is illustrated in FIG. 3 for template 382 with primer binding site 381 attached to solid phase support 380. Primer 384 and DNA polymerase 386 operably bound to template 382. Upon the addition 388 of dNTP (shown as dATP), polymerase 386 incorporates a nucleotide since “T” 385 is the next nucleotide in template 382. Wash step 390 follows, after which the next dNTP (dCTP) is added 392. Optionally, after each step of adding a dNTP, an additional step may be performed wherein the reaction chambers are treated with a dNTP-destroying agent, such as apyrase, to eliminate any residual dNTPs remaining in the chamber, which may result in spurious extensions in subsequent cycles.

In one embodiment, the sequencing method exemplified in FIG. 3 may be carry out using the apparatus of the present teaching in the following steps: (a) disposing a plurality of template nucleic acids into a plurality of reaction chambers disposed on a sensor array, the sensor array comprising a plurality of sensors and each reaction chamber being disposed on and in a sensing relationship with at least one sensor configured to provide at least one output signal representing a sequencing reaction byproduct proximate thereto, and wherein each of the template nucleic acids is hybridized to a sequencing primer and is bound to a polymerase; (b) introducing a known nucleotide triphosphate into the reaction chambers; (c) detecting incorporation at a 3′ end of the sequencing primer of one or more nucleotide triphosphates by a sequencing reaction byproduct if such one or more nucleotide triphosphates are complementary to corresponding nucleotides in the template nucleic acid; (d) washing unincorporated nucleotide triphosphates from the reaction chambers; and (e) repeating steps (b) through (d) until the plurality of template nucleic acids are sequenced.

For embodiments where hydrogen ion is measured as a reaction byproduct, the reactions further should be conducted under weak buffer conditions, so that the hydrogen ions react with a sensor and not extraneous components (e.g., microwell or solid supports that may have surface buffering capacity) or chemical constituents in particular pH buffering compounds. In one embodiment, a weak buffer allows detection of a pH change of at least ±0.1 in said reaction chamber, or at least ±0.01 in said reaction chambers, or at least ±0.001 in said reaction chambers, or at least ±0.0001 in said reaction chambers, or in some embodiments less than ±0.0001 in said reaction chambers.

Several potential sources of noise may affect output signals from sensors when a large number of electrochemical reactions are carried out in a microwell array integrated with a sensor array, such as described by Rothberg et al. (cited above). Such sources of noise include thermal sensitivity of the sensors, electrical potential disturbances in the fluid (such as resistive or thermal noise in the fluids, reference voltage changes due to different fluids contacting the reference electrode, and the like) and pH changes due to bulk changes in fluids that are passed over the sensor array (referred to herein as “reagent change noise”).

Another source of noise may arise when successive reagent flows pass over a sensor array (i.e., reagent change noise). The magnitude of such noise depends on several factors including the nature of the measurement being made (e.g., pH, inorganic pyrophosphate (PPi), other ions, or the like) whether a leading or trailing reagent in a reagent change has a property or constituent, e.g. pH, which affects sensor performance and the magnitude of the influence, the relative magnitude of the reagent change effect in comparison with the reaction signal being monitored, and so on. The present methods alleviate noise and improve sequencing performance

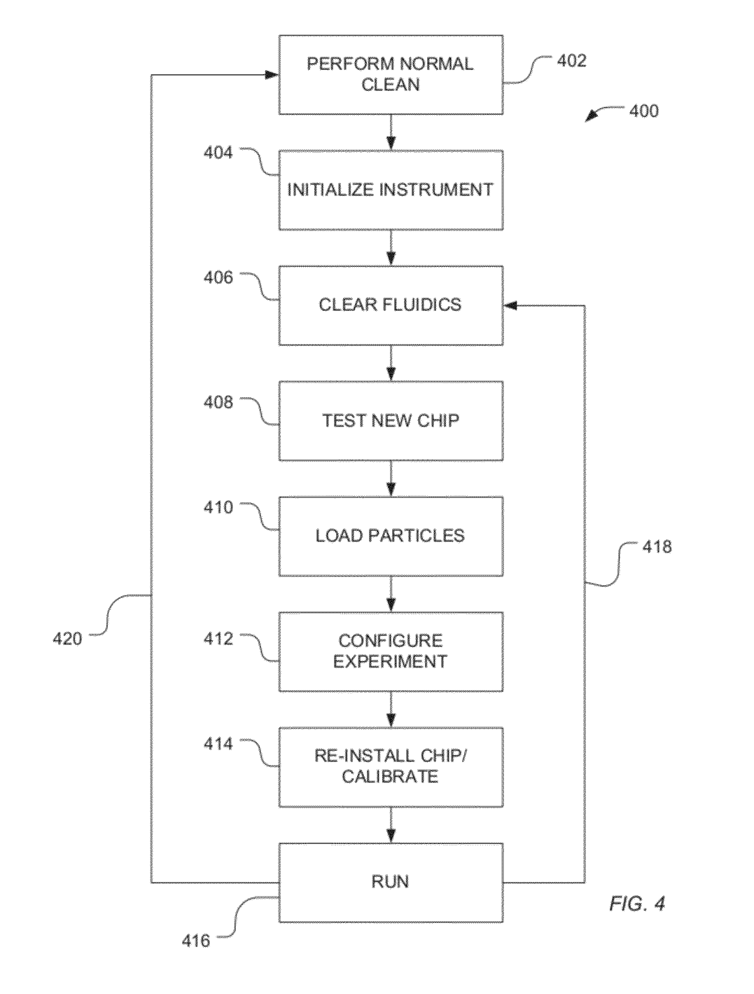

FIG. 4 illustrates an exemplary workflow 400 with steps that may be used to prepare a flow cell and sensor array 100 for a sequencing run or control run as well as runtime operational modes. Various operations provided in the workflow introduce liquid reagent into the flow cell and sensor array 100 and are expected to have been pre-calibrated with pH values adjusted to selected and generally narrow or precise ranges. For example, wash/detergent solutions and nucleotide containing reagent solutions may have specified pH ranges of approximately 7.5±0.1.

As illustrated in FIG. 4, a method 400 can be performed to operate a sequencing instrument. For example, the sequencing instrument can be cleaned, as illustrated 402. Such cleaning can include running cleaning solutions through the fluidics of the instrument to remove byproducts, residue and other reagents from the fluidics or can include removing disposable portions of the sequencing instrument, such as tubes and sequencing chips.

The instrument can be initialized, as illustrated at 404. For example, a routine on the instrument can be activated to check various sensors and contacts within the device for operability.

As illustrated 406, the fluidics of the instrument can be cleared, such as using an inert gas. In a particular sequencing instrument, a chip can be installed and tested, as illustrated at 408. For example, the chip can include an array of sensors, such as ion sensors, disposed on a common substrate. A testing routine can be implemented. In one example, the testing routine tests a dry chip. In another example, the chip may be tested by flowing a reagent or a wash solution over the chip and testing the operability of the chip under wet conditions.

As illustrated at 410, polynucleotide-containing particles can be loaded into wells of a sensor array, such as a sensor array having ion sensors disposed on a common substrate. Optionally, loading is performed while the chip is in position on the instrument. In another example, the chip can be removed from the instrument, loaded, and returned to the instrument. Alternatively, the polynucleotide-containing particles can be loaded prior to initially loading the chip. In a particular example, the experiment is configured within the instrument, as illustrated at 412, and if the chip is removed from the instrument, the chip can be reinstalled on the instrument and calibrated, as illustrated at 414.

As illustrated at 416, an experiment can be run. For example, a sequence of nucleotide solutions, each including a unique nucleotide type, can be applied to the chip to perform a sequencing operation. Between each nucleotide solution, a wash solution can be applied. Once sequencing is performed, the same chip having the same loaded polynucleotide-containing particles can be retested using the same or different protocols, as illustrated at 418. Alternatively, if the experiment is complete, the instrument can be cleaned and prepared for a new run, as illustrated 420.

If the instrument includes a separate pH sensor, pH adjustment of solutions and reagents can be carried out during instrument initialization or fluidics cleaning, as illustrated at 404-406. Alternatively, an ion sensing array of a sequencing chip can be utilized to detect the pH of solutions and reagents. In such an example, pH adjustment of wash solutions or nucleotide solutions can be performed either during chip testing, as illustrated at 408, prior to loading, or can be performed following loading, such as during the configuration of the experiment, as illustrated at 412, or the calibration, as illustrated at 414.

In a particular example, a sequencing system is prepared by cleaning a sequencing instrument and preparing solutions and reagents for use by instrument. Solutions can include wash solutions and solutions including select nucleotides. In a particular example, a reagent is included that can be used to adjust the pH of solutions in a positive direction, making the solutions more basic. In another example, a reagent can be included that the decreases pH of a solution, rendering the solution more acidic. In a further example, the system can include both a reagent that increases pH and a reagent that decreases pH. Once the solutions and reagents are prepared, they can be coupled to the sequencing instrument. In an example, air can be purged from the solutions and reagents and the reagent containers and various fluid lines leading to and from the reagent containers can be checked for leaks. The fluid lines can be flushed with a wash solution. The pH of one or more solutions can be adjusted by the instrument.

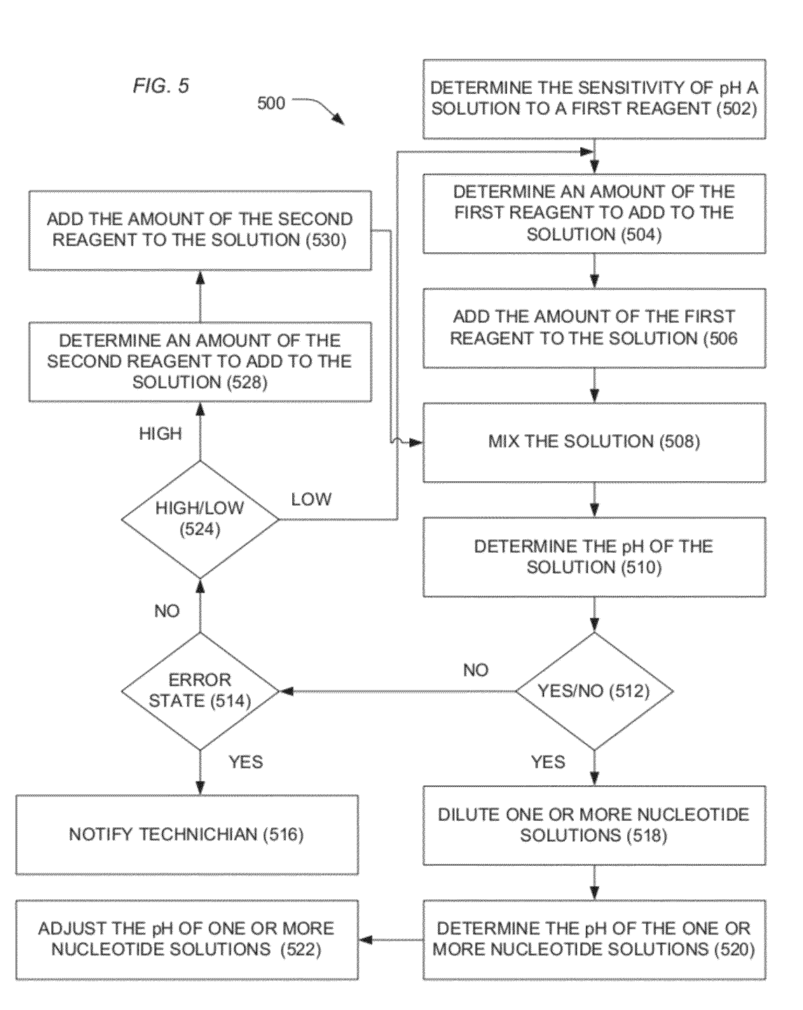

In an example illustrated in FIG. 5, a method 500 includes determining the sensitivity of a pH of a solution to a first reagent, as illustrated at 502. In an example, determining the sensitivity of the pH of the solution can include determining an initial pH of the solution, adding an amount of the first reagent to the solution, mixing the solution, and testing the pH of the solution to determine a change from an initial pH.

The pH can be determined by a pH sensor. Alternatively, particularly when the sequencing instrument includes a sequencing component (e.g., sequencing chip) that is sensitive to changes in ion concentration, the pH can be determined using an array of ion sensors of the sequencing component. In particular, the sequencing component can include an array of ion sensitive field effect transistor (ISFET) devices disposed on a common substrate within a flow cell.

Based on the sensitivity of the pH of the solution to the first reagent, an amount of the first reagent to be added to the solution to approach a target pH can be determined, as illustrated at 504. Depending upon the nature of the solution, the sensitivity can be modeled as a linear model and an amount of reagent can be determined based on a projection of the linear model. In another example, the solution can be considered a buffered solution and a model incorporating buffering behavior can be utilized to determine an unexpected amount of the first reagent to add to the solution to approach a target pH. Alternatively, the sensitivity can be scaled using a scaling factor that depends on the nature of the solution or the history of the sensitivity. The amount of the first reagent can be the product of the sensitivity or the scaled sensitivity times the difference in pH between a target pH and the pH of the solution.

The determined amount of the first reagent can be added, as illustrated 506, and the solution can be mixed, as illustrated 508. In an example, the reagent can be mixed using a magnetic stir bar, a sonicator, or other mechanical means. In an alternative example, the reagent solution can be mixed using gas bubbles, in particular inert gas bubbles.

As illustrated 510, the pH of the solution can be tested. For example, the pH can be tested using a pH sensor or alternatively using the array of ion sensitive sensors of the sequencing component. As illustrated 512, when the pH of the solution is determined to be within a desired range or close to a target pH, the solution, such as a wash solution, for example, can be used to dilute one or more nucleotide solutions, as illustrated at 518. Optionally, the nucleotide solution can be tested to determine the pH of the nucleotide solution, as illustrated 520 and, in an example, the pH of the nucleotide solution can be adjusted, as illustrated at 522, utilizing a method similar to method steps of 502-512. Alternatively, the pH of the nucleotide solution can be measured and an error state established when the pH of the nucleotide solution falls outside of a desired range.

When the tested solution is not within a desired range or close to the target pH, the system can test for an error state, as illustrated 514. For example, an error state may be determined when an excess amount of reagent fails to provide the solution with a pH within a target range or close to a target pH. In another example, the error state may be indicated when too many adjustments are made and the desired pH range or proximity to the target pH is not achieved. In a further example, an error state may be indicated when the pH is beyond a target pH, such by an amount more than expected.

In an example, the pH procedure can be configured to flag conditions where there are significant variances from expected conditions. For example, when adjusting the pH of a solution, an excess amount of utilized reagent may indicate that there is a problem with the reagents themselves and the system can be set up to provide a notice or alarm to the operator. Using convention manual methods an operator might overlook, ignore, or not appreciate that excessive pH adjustment is a potential issue or suggests a problem with the reagents. Thus, the system of the present teachings can help an operator avoid or correct unsatisfactory or problematic conditions which might otherwise go uncorrected or unnoticed.

When an error state does not exist and the pH of the solution is not in proximity to the target pH, a process for adjusting the pH can be repeated. In particular, the process can include determining an amount of the first reagent add to the solution, adding the amount of the first reagent, and retesting the solution. In a particular example, the system can determine whether the pH of the solution is too high or too low, as illustrated at 524. In an example in which the first reagent is to increase the pH of the solution, an amount of the first reagent can be determined when the pH of the solution is low. Alternatively, when the pH of the solution is determined be high, an amount of a second reagent to reduce the pH of the solution can be determined, as illustrated at 528 and the amount of the second reagent can be added to the solution, as illustrated at 530. Subsequently, the solution can be mixed and tested, as illustrated at 508-510.

Each of the solutions and reagents can be provided to the instrument prior to adjusting the pH of the solution. Alternatively, the nucleotide solutions can be installed following pH adjustment of the wash solution as above. After installation, purging, and leak checking, the nucleotide solution bottles can be filled to dilute the nucleotide solutions with a wash solution. Fluid lines from the nucleotide solutions can be primed and the pH of the nucleotide solutions can be tested.

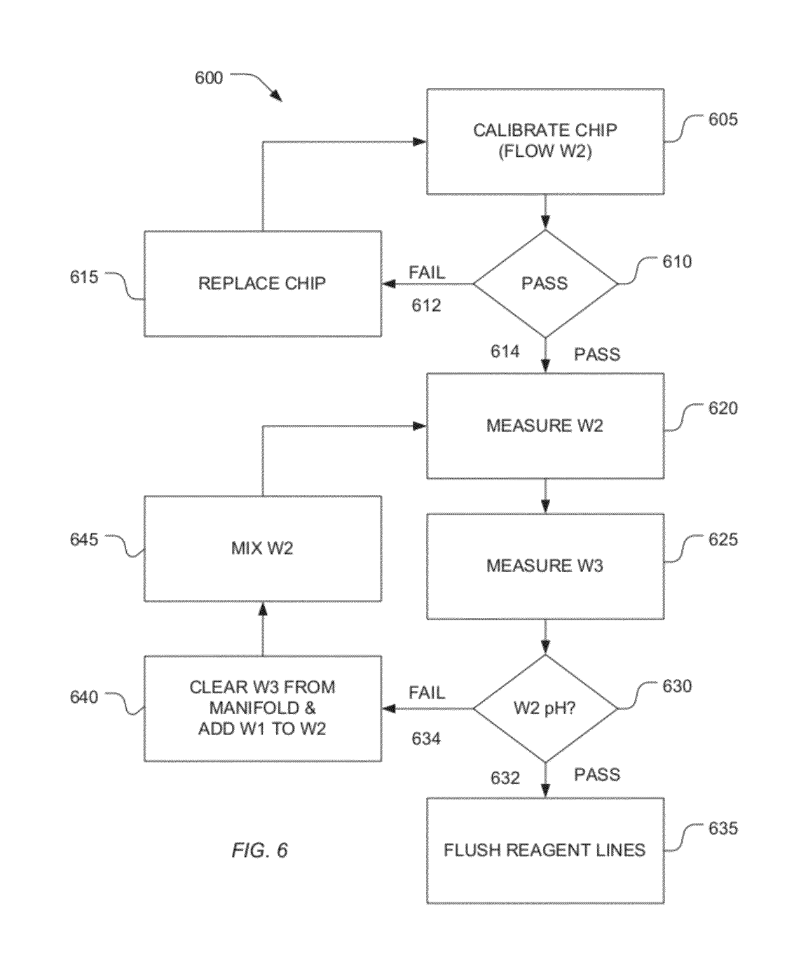

The methods described herein may be readily initiated at selected times throughout the instrument's operation without having to withdraw the solutions from the system directly and thus can be performed “on-the-fly” as needed or desired. In the automated procedure, pH detection and adjustment may be performed directly on the sequencing instrument using a pre-programmed calibration logic or algorithm 600 as shown in FIG. 6. Here wash solution 1 may be a solution used to adjust pH, wash solution 2 may be a solution whose pH is to be adjusted, and wash solution 3 may be a reference solution whose pH may be known or within an expected range. Other solutions for pH modification may be applied without departing from the scope of the present teachings.

According to the method 600 of FIG. 6, the procedure may start at state 605 where a flowcell/chip calibration routine is invoked. In this state, wash solution 110 may be introduced into the flowcell 100 in desired amounts directed by the fluidics controller 116 and valve block assemblies 116, referencing FIG. 1. The instrument is programmed in check state 610 to determine if the calibration passes where: (a) if the calibration fails 612 a fault condition is registered and the flowcell/chip is requested to be replaced in state 615 or (b) if the calibration passes 614 then the pH of the wash solutions are measured in states 620 and 625. The pH measurement of the wash solutions is accomplished on-instrument using the same or similar components as is used to measure nucleotide incorporation events. In various embodiments, because the sequencing system is capable of providing extremely precise and accurate pH measurements to detect and register changes in pH resultant from nucleotide sequencing reactions, the system is also well suited to measure pH of wash and reagent solutions where the pH measurement requirements are less rigorous. Furthermore, use of the system as a liquid reagent pH sensing apparatus desirably provides a large dynamic range with high precision/accuracy throughout the range. While the sensor array typically is configured with many sensors (e.g. millions) only a portion (as little as one) of the sensors need be used for pH determination of solutions. Use of a reduced sensor subset may result in less data being generated or the ability to more rapidly acquire the data. Thus in one exemplary embodiment, a sensor region of approximately 100×100, 250×250, 500×500 sensors or any other combination may be used in measuring the solution pH. Such sensors may be collocated in a region with respect to one another or selected from different positions or regions within the flowcell. In various embodiments, the instrument may be standardized on a pixel region (e.g. 500×500 or some other combination) in the approximate middle of the sensor array. Furthermore, multiple sensor readings may be compared to one another prior to registering the solution pH where if there are discrepancies between sensor readings above a selected threshold then the pH measurements may be reacquired before accepting.

In one aspect, pH adjustment of the wash or reagent solutions may be accomplished directly on the instrument by introducing desired amounts of one wash or reagent solution at a selected pH into another wash or reagent solution at a different pH. The instrument measures the pH of the solution being tested and makes calculations as to how much of another wash or reagent solutions should be added to bring the pH of the test solution within a desired range. The existing solutions, fluidic control and valving of the instrument may then be used to direct desired solutions within the instrument and add one solution to another target solution to achieve the desired pH. On instrument mixing may further be achieved by introducing gas bubbles into the appropriate containment vessels, creating fluid flow within the appropriate containment vessels by sequential removal and introduction of liquid through the fluidic lines, by waiting a period of time for pH equilibration to occur, or by other such methods.

In various, embodiments, the instrument may apply a dynamic estimation routine to aid in reaching the desired pH without significant overshooting or undershooting. In one exemplary routine, the pH of test solution may be measured following the addition of a relatively small known amount of acidic or basic solution to the test solution. Based on the overall change of pH in the test solution resulting from this addition, the instrument may calculate the actual amount of acidic or basic solution to achieve a desired target pH of the test solution. The amount of acidic or basic solution may then be added to achieve the target pH without multiple iterations of under or overshooting. In another exemplary embodiment, the target acidic or basic solution addition volume or amount may be set slightly below that which would be needed to achieve the target pH (for example between approximately 90-95% of target pH). This approach may be useful to bring the test solution quickly within range of the desired target pH where further small additions of acidic or basic solution may then be added to achieve the target pH readily.

Returning to the method 600, a check state 630 determines if the wash solution has achieved the desired pH and (a) if yes 632, exits the method in state 635 where the reagent lines in the system are flushed and prepared for instrument runtime operation or (b) if no 634, proceeds to state 640 where the manifold and reagent delivery lines are cleared of wash reagent and additional pH adjusting solution is added to the wash solution to be mixed in state 645 and re-measured to determine the wash solution pH in state 620.

The methods for automated pH adjustment are not limited to any particular solution. For example, while discussed in the context of wash solution measurement and pH adjustment, such methods may also be readily applied to other solutions present in the system without departing from the scope of the present teachings. Likewise, the use of pH adjusting solutions may be flexibly configured on the basis of what types of solutions are utilized with the instrument, their typical pH, and there compatibility for use as a pH modifier to other solutions. Furthermore, the exemplary method 600 may be altered in various ways to achieve similar objectives of measuring and adjusting selected solutions pH.

In a particular example, a reagent is added to a solution using an iterative process that determines subsequent amounts of reagent to be add to the solution based on a history of sensitivity of the pH of the solution to addition of the reagent. In an example, sensitivity is the ratio of the amount of reagent added to the solution relative to the change in pH.

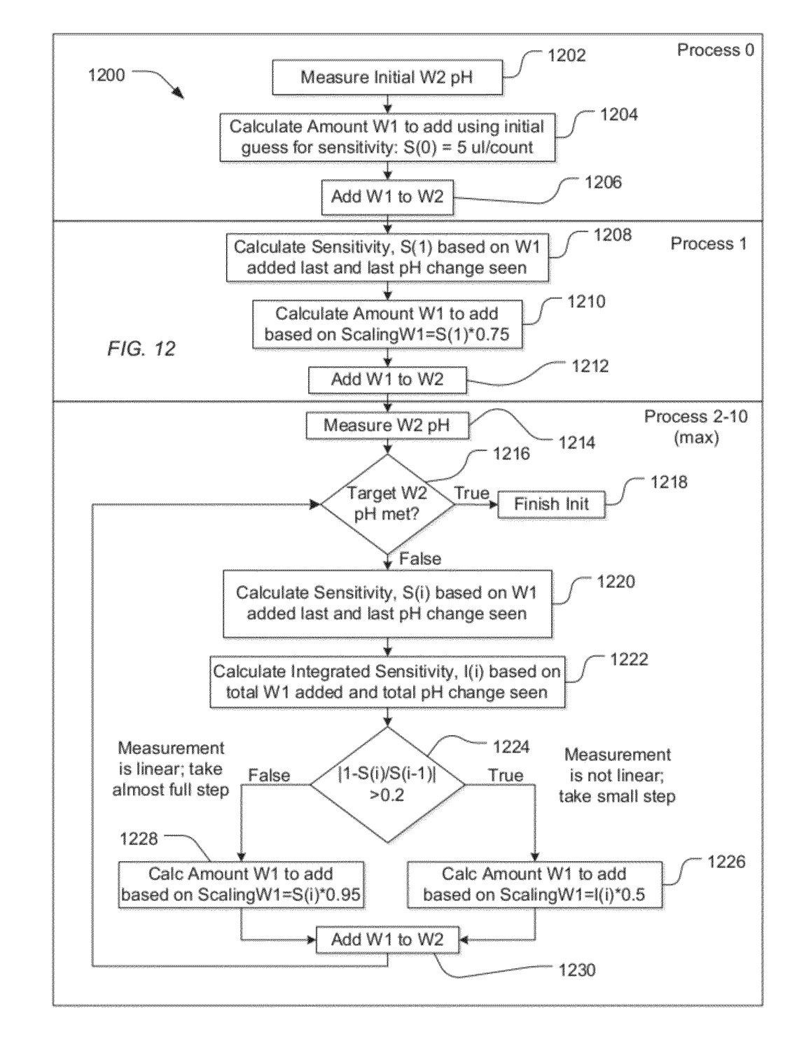

For example, a method 1200 illustrated in FIG. 12 includes a Process 0 that includes measuring an initial pH of a W2 solution, as illustrated at 1202. In this example, the pH of a wash solution, designated as W2, is adjusted using a reagent, designated as W1. As above, the pH can be measured on a pH meter integral with the instrument or can be measured on an array of ion sensitive sensors disposed on a sequencing component.

Based on the initial pH of the W2 solution, the system calculates an amount of W1 reagent to add to the W2 solution using an initial guess for sensitivity, as illustrated at 1204. For example, the initial sensitivity estimate can be 5 pm/count(pH). Alternatively, other initial sensitivity values can be utilized depending on the nature of the W2 solution. The amount of W1 reagent to be added to the W2 solution is the product of the sensitivity estimate and the difference between the measured pH of the W2 solution and the target pH. As illustrated at 1206, the calculated amount of the W1 reagent can be added to the W2 solution. The solution can be mixed and the pH again measured.

Subsequently, Process 1 is performed in which a subsequent sensitivity is calculated based on the previously added amount of W1 reagent and the last observed pH change, as illustrated at 1208. An amount of W1 reagent to be added to the W2 solution can be calculated based on a scaled sensitivity and the difference between the pH of the W2 solution and a target pH, as illustrated at 1210. For example, the scaled sensitivity can be 75% of the calculated sensitivity for Process 1, denoted S(1) in FIG. 12. As illustrated at 1212, the scaled amount of W1 reagent can be added to the W2 solution, and the W2 solution can be mixed. The selected scale (e.g., 75%) can be selected based on aspects of the system, the W1 reagent, and the W2 solution and can be more than 75% or less than 75%.

Following Process 1, an iterative set of processes can be performed. As illustrated, the process can be performed more than one time. For example, the process can be repeated up to 9 times (Processes 2-10), as illustrated in FIG. 12. However, depending on the nature the system, a maximum other than 10 process iterations can be selected. For example, the maximum can be set to less than 10, such as less than 8, less than 6, or less than 4. In another example, the maximum can be greater than 10, such as at least 12.

As illustrated at 1214, the pH of the W2 solution can be measured. As illustrated at 1216, the measured pH of the W2 solution can be compared with a target pH. When the W2 solution has a pH approximate the target pH, the process ends, as illustrated at 1218.

When the pH of the W2 solution does not approximate the target pH, a sensitivity can be calculated, denoted S(i) for iteration i (i.e., Process i). The sensitivity can be calculated based on the previously added amount of W1 reagent and the observed change in pH, as illustrated at 1220.

Optionally, the system can also determine an integrated sensitivity, denoted by I(i) for each Process i. The integrated sensitivity can be determined based on the total amount of W1 reagent added to the W2 solution and the total observed change in pH of the W2 solution, as illustrated at 1222.

Based on the nature of the change in sensitivity between iterations or the history of sensitivity, the system can determine whether the change in pH is performing in a linear fashion or a nonlinear fashion. In particular, when the sensitivity exhibits a significant change, it is likely that the pH of the W2 solution is not changing in a linear fashion. For example, as illustrated 1224, when the absolute value of 1 minus the ratio of the present sensitivity to the previous sensitivity is greater than 0.2 (|1−S(i)/S(i−1)|>0.2), the pH of the W2 solution is not behaving linearly. When the pH does not behave linearly, smaller steps are taken in the iterative process to approach the target pH. For example, as illustrated at 1226, the amount of W1 reagent to be added to the W2 solution can be calculated using a scaling factor significantly less than 1. For example, a scaling factor can be less than 60%, such as 50% or less. Further, the amount of W1 reagent to be added to the W2 solution can be determined based on integrated sensitivity, I(i), determined at 1222. Alternatively, the present sensitivity S(i) can be used. As illustrated in FIG. 12, the amount of the W1 reagent to be added to the W2 solution can be, for example, the determined as the product of 50% of the integrated sensitivity I(i) times the pH difference between the target pH and that of the W2 solution.

On the other hand, when the pH change behaves linearly, larger steps towards the target pH can be taken. For example, the amount of W1 reagent to be added to the W2 solution can be scaled using factors close to 1. For example, the factor may be at least 80%, such as at least 90%, or even at least 95%. Further, the calculation can be, for example, based on the current sensitivity S(i) for the current process step. As such, as illustrated at 1228 of FIG. 12, the amount of W1 reagent to be added to the W2 solution can be, for example, the product of 95% of the current sensitivity times the difference in pH between the W2 solution and the target pH.

Once the amount of W1 is determined, the amount of W1 reagent can be added to the W2 solution, as illustrated at 1230. Following stirring and equilibrium, the pH of the W2 solution can be measured, and again, it can be determined whether the pH of the W2 solution approximates the target pH, as illustrated 1216. When the pH of the W2 solution approximates the target pH, the process can be stopped, as illustrated at 1218. When the process iterates too many times, as indicated by the maximum Process number, the process can be stopped and the user notified that an error may exist within the system or the reagents.

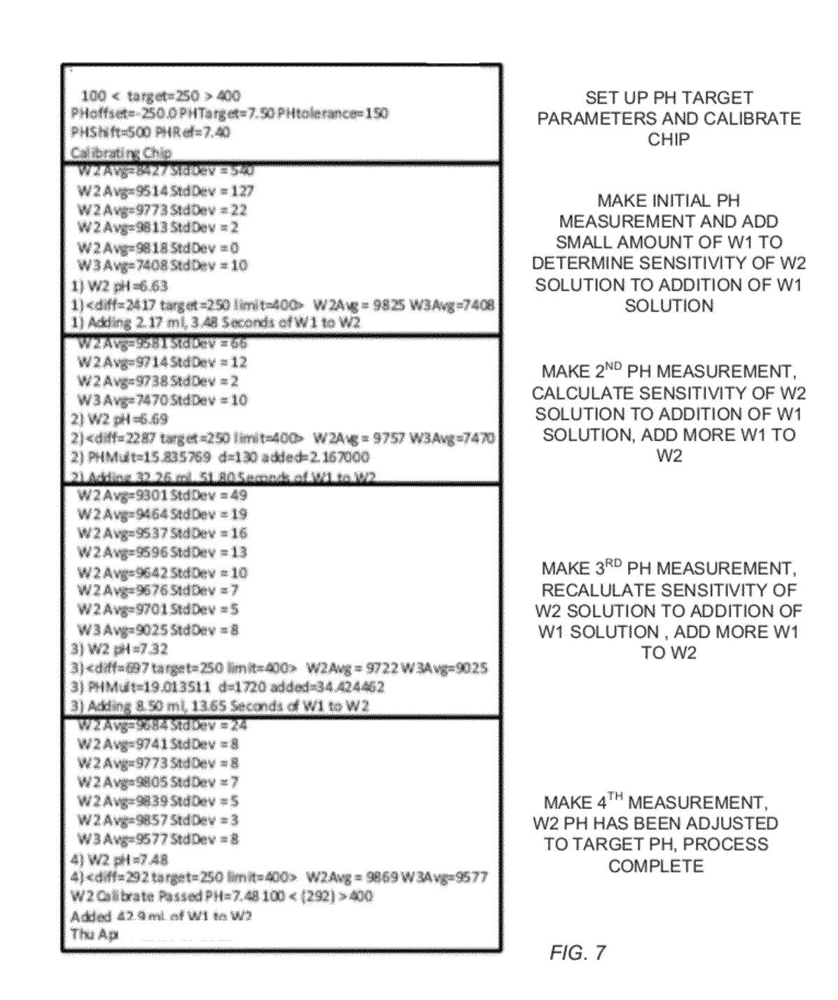

As shown in FIG. 7, the system may further be configured to provide an output report or log of various aspects of the pH adjustment functionality. Such a log may be useful to the user to understand what changes were made to the solutions, what pH measurements were obtained, and for purposes of quality control, troubleshooting, validation, and the like. In one aspect, the log may provide real time or semi-real time feedback to the user as to what operations are being performed during the pH adjustment cycles and generate statistics useful to the user to monitor whether the instrument is operating within desired parameters.

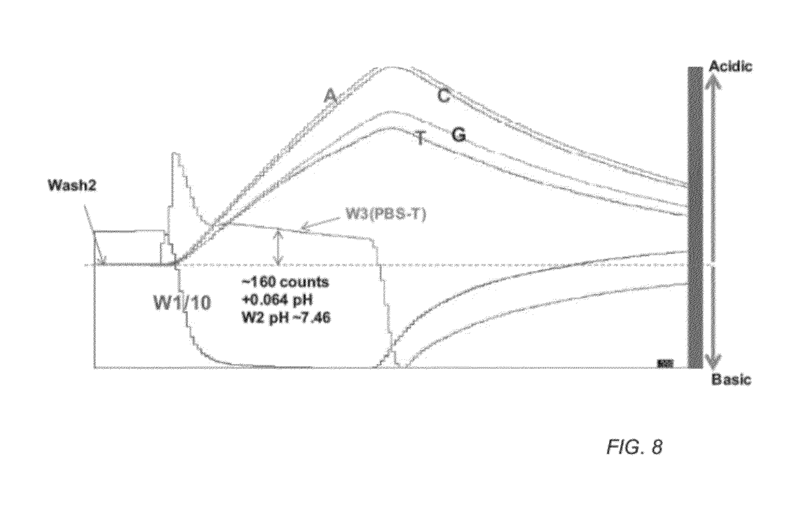

FIG. 8 provides an exemplary visual output of runtime pH changes for various solutions within the instrument which likewise can be helpful to the user to verify proper instrument operation. The pH adjustment functionality allows the user to visualize the pH of the various solutions throughout the course of a run which can be useful for purposes of quality control, troubleshooting, validation, and the like.

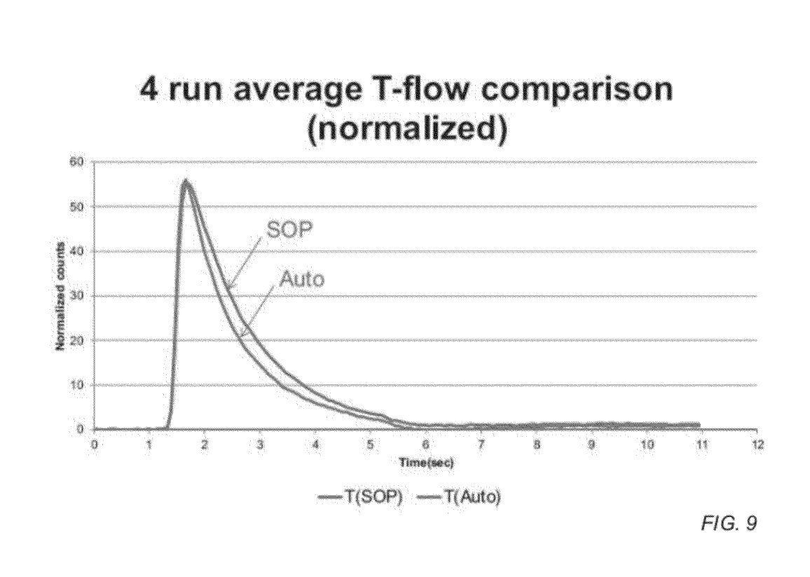

FIG. 9 demonstrates the ability of the pH adjustment functionality to provide improved runtime response as compared to manually adjusted solution pHs. As illustrated in FIG. 9, the instrument response (T(Auto)) is better than the response (T(SOP)) to the manual method thus indicating the viability of replacing the manual methods with those that have been automated.

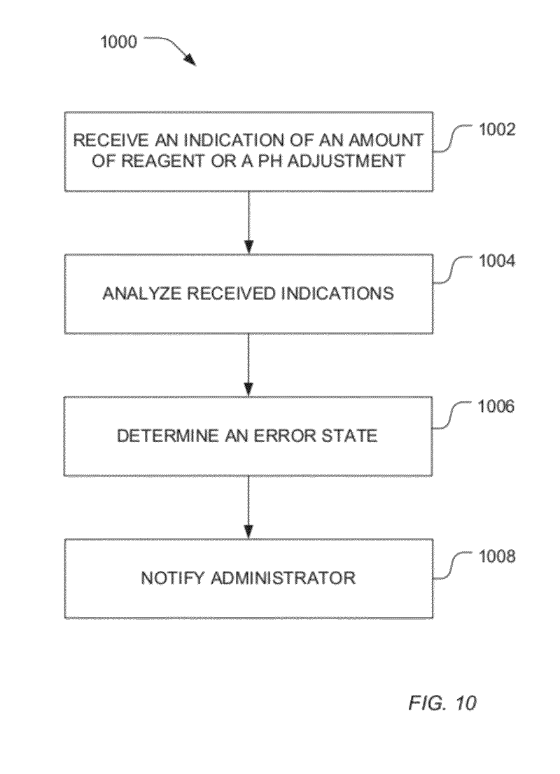

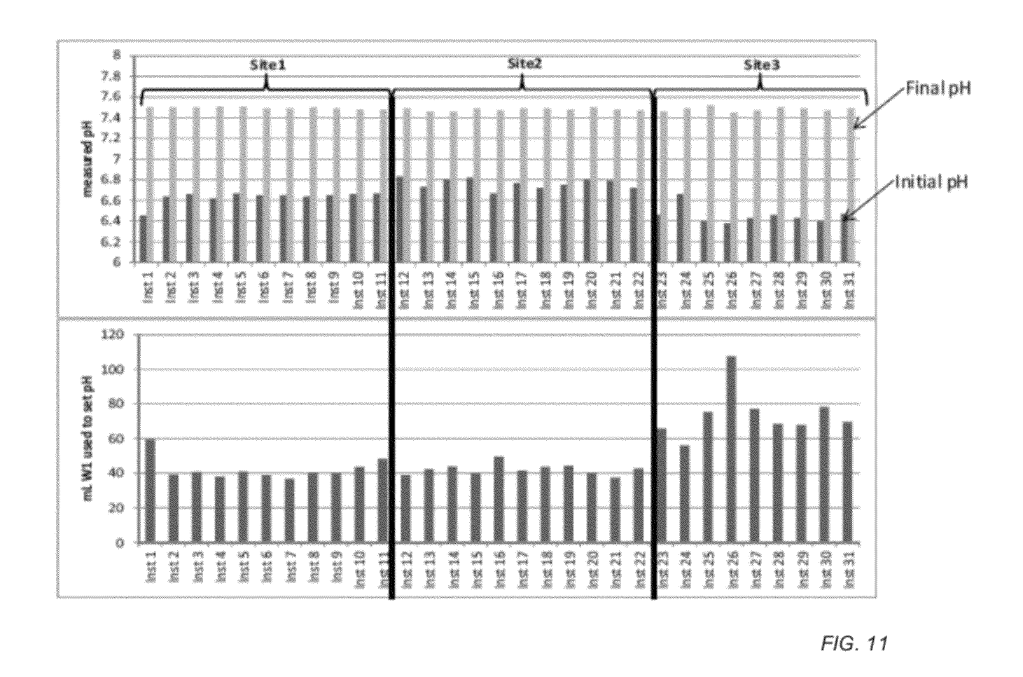

As illustrated FIG. 10, a method 1000 can be used to monitor the performance of one or more instruments or monitor the performance of several instruments at one or more sites remote from each other. Such sites can be remote sites, for example, located in different rooms, located on different floors of a building, or located in different geographic regions. In another example, such sites can be managed by different technicians. In the method 1000 illustrated in FIG. 10, the amount of one or more reagents used to adjust the pH or the resulting adjustment on each of a plurality of instruments can be received at a central location, such as a server system in communication with each of the sites. For example, FIG. 11 illustrates collecting indications from one or more instruments at each of three sites. In an example, the indication can include initial and final pHs of a wash solution, differences between the initial and final pHs of the solution, or an amount of one or more reagents used to manipulate the pH of the wash solution.

As illustrated at 1004, the received indications can be analyzed to determine differences in performance between machines, sites, or personnel. FIG. 11 demonstrates an exemplary use of the pH methods to identify potential quality issues. The starting or initial pH of the wash reagent and amount of the pH adjusting reagent appears significantly different at Site 3 than the other sites. Such information can be used to “flag” instrument or site specific problems. For example, based on the aforementioned data, service personnel are able to identify a problem and trace the problem to its origin. In such an example, personnel at Site 3 may have failed to perform some steps in the wash solution or reagent preparation. Consequently, the improper reagent preparation may have allowed atmospheric CO₂ to acidify the wash reagent prior to installation on the instrument. The reporting functionality desirably provides a way to identify such sources of error or variance and allows correction in an efficient and reliable manner.

In the example illustrated in FIG. 11, Site 3 indicates that a large amount reagent is used to adjust the pH of the machines at Site 3 relative to the machines at other sites. Such a large adjustment can indicate procedural errors followed by technicians at Site 3, problems with water purification systems at Site 3, the presence of atmospheric carbon dioxide during solution preparation, or a combination thereof In a further example, specific machines within a site can be monitored for performance relative to other machines. For example, a first machine at Site 1 utilizes a greater adjustment than other machines collocated at Site 1. As such, error statuses, such as machine levels error status, a site level error status, or system wide error status, can be determined, as illustrated at 1006.

Once an error status is determined, an administrator can be notified, as illustrated 1008. For example, the notice can be provided in an interface to a database software. In another example, the notice can be provided by e-mail, text messaging, a phone call with an automated voice system, or other communication means.

As indicated in this diagram the complexity and manual operations of the startup procedure are significantly reduced using the automated pH procedure where the instrument performs the fine adjustments of pH required for operation. Also such an automated approach reduces or eliminates errors associated with manual operations that can be introduced as a result of the operator technique, systematic error or accuracy flaws in the pH measuring device, or pH drift over time compared to previous pH measurements of the reagents.

In an example, the pH procedure can be configured to flag conditions where there are significant variances from expected conditions. For example, when adjusting the pH of a solution, an excess amount of adjustment is utilized may indicate that there is a problem with the reagents themselves and the system can be set up to provide a notice or alarm to the operator.

For a pH adjustment run, the system may be configured with an expected range of pH addition to the various solutions. If the pH adjustment method is able to be performed within this range, the system may proceed to a ready state. If however, excess adjustment is utilized, an error condition, flag, or notice may be set. Such a condition may be suggestive of a problem with one or more of the reagents and thus require additional corrective measures be taken. Thus the pH adjustment system is able to help the operator monitor the “health” of various reagents in a convenient manner.

The pH of solutions is conventionally manually measured and adjusted prior to loading on the instrument. Because of the relatively tight pH requirements, adjustment of each liquid reagent's pH may require multiple repeated measurements and base/acid additions in small increments to insure the desired value is achieved. Such processes may be time consuming, tedious and error prone when using manual methods. According to the present teachings, such drawbacks are significantly overcome and instrument preparation facilitated by adaptation of methods for pH adjustment as described in greater detail above. In particular, it has been discovered that the present techniques result in low pH variance, leading to better pH control and better sequencing performance.

In a first aspect, a method of preparing a sequencing device includes determining a sensitivity of a pH of a solution to a first reagent, determining an amount of the first reagent to add to the solution to approach a target pH, adding the amount of the first reagent to the solution, and diluting a nucleotide solution with the solution.

In an example of the first aspect, determining the sensitivity of the pH of the solution to the first reagent includes determining an initial pH of the solution. In an example, determining the initial pH of the solution includes determining the initial pH with an array of ion sensitive components disposed on a common substrate. For example, the array can form a portion of a sequencing device. In a further example, determining the sensitivity further includes adding a first amount of a first reagent to the solution following determining the initial pH of the solution. In an additional example, determining the sensitivity further includes determining a second pH of the solution following adding the first amount of the first reagent.

In another example of the first aspect and the above examples, determining the amount of the first reagent includes determining a scaled sensitivity. For example, the scaled sensitivity can be determined based on a sensitivity history.

In a further example of the first aspect and the above examples, the method further includes determining a pH of the nucleotide solution. For example, the method further includes adjusting the pH of the nucleotide solution in response to determining the pH of the nucleotide solution.

In an additional example of the first aspect and the above examples, the method further includes adding an amount of a second reagent to the solution, the second reagent having a pH different from the first reagent.

In another example of the first aspect and the above examples, the method further includes determining a pH of the solution following adding the amount of the first reagent to the solution. For example, the method can further include determining an error state based on determining the pH of the solution. In another example, the method can further include determining a second amount of the first reagent to add to the solution.

In a further example of the first aspect and the above examples, the first solution is a wash solution of the sequencing device.

In a second aspect, a method of preparing a sequencing device includes determining an initial pH of a solution, adding a first amount of a first reagent to the solution, determining a pH of the solution, determining a second amount of the first reagent to add to the solution, adding the second amount of the first reagent to the solution, determining the pH of the solution, and diluting a nucleotide solution with the solution.

In an example of the second aspect, determining the initial pH of the solution includes determining the initial pH with an array of ion sensitive components disposed on a common substrate. For example, the array can form a portion of a sequencing device.

In another example of the second aspect and the above examples, determining the second amount of the first reagent includes determining a scaled sensitivity. For example, the scaled sensitivity can be determined based on a sensitivity history.

In a further example of the second aspect and the above examples, the method further includes determining a pH of the nucleotide solution. For example, the method can further include adjusting the pH of the nucleotide solution in response to determining the pH of the nucleotide solution.

In an additional example of the second aspect and the above examples, the method can further include adding an amount of a second reagent to the solution, the second reagent having a pH different from the first reagent.

In a third aspect, a method of protocol monitoring includes from each of a plurality of sequencing devices, receiving an indication of an amount of a reagent added to a solution to adjust the pH of the solution to within a range of a target pH, determining an error state based at least in part on the received indications, and notifying an administrator of the determined error state.

In an example of the third aspect, determining the error state includes determining that an amount of adjustment is in excess. For example, determining the error state can include determining a site level error.

In a fourth aspect, a system includes a solution having a first pH, a first reagent with a second pH greater than the first pH, a pH analyzing component configured to measure the pH of the solution, a pH calculating component configured to determine an amount of the first reagent to be added to the reagent to achieve a selected pH for the solution, and a pH modifying component configured to direct a selected amount of the reagent into the solution so as to achieve the selected pH.

In an example of the fourth aspect, the pH analyzing component comprises a sequencing chip.

In another example of the fourth aspect and the above example, the system further includes a second reagent having a third pH, the third pH being less than the first pH.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

1. A method of preparing a sequencing device, the method comprising:
determining a sensitivity of a pH of a solution to a first reagent;
determining an amount of the first reagent to add to the solution to approach a target pH;
adding the amount of the first reagent to the solution; and
diluting a nucleotide solution with the solution.

2. The method of claim 1, wherein determining the sensitivity of the pH of the solution to the first reagent includes determining an initial pH of the solution.

3. The method of claim 2, wherein determining the initial pH of the solution includes determining the initial pH with an array of ion sensitive components disposed on a common substrate.

4. The method of claim 3, wherein the array forms a portion of a sequencing device.

5. The method of claim 2, wherein determining the sensitivity further comprises adding a first amount of a first reagent to the solution following determining the initial pH of the solution.

6. The method of claim 5, wherein determining the sensitivity further comprises determining a second pH of the solution following adding the first amount of the first reagent.

7. The method of claim 1, wherein determining the amount of the first reagent includes determining a scaled sensitivity.

8. (canceled)

9. The method of claim 1, further comprising determining a pH of the nucleotide solution.

10. (canceled)

11. The method of claim 1, further comprising adding an amount of a second reagent to the solution, the second reagent having a pH different from the first reagent.

12. The method of claim 1, further comprising determining a pH of the solution following adding the amount of the first reagent to the solution.

13. (canceled)

14. (canceled)

15. The method of claim 1, wherein the first solution is a wash solution of the sequencing device.

16. A method of preparing a sequencing device, the method comprising:
determining an initial pH of a solution;
adding a first amount of a first reagent to the solution;
determining a pH of the solution;
determining a second amount of the first reagent to add to the solution;
adding the second amount of the first reagent to the solution;
determining the pH of the solution; and
diluting a nucleotide solution with the solution.

17. The method of claim 16, wherein determining the initial pH of the solution includes determining the initial pH with an array of ion sensitive components disposed on a common substrate.

18. The method of claim 17, wherein the array forms a portion of a sequencing device.

19. The method of claim 16, wherein determining the second amount of the first reagent includes determining a scaled sensitivity.

20. (canceled)

21. The method of claim 16, further comprising determining a pH of the nucleotide solution.

22. (canceled)

23. The method of claim 16, further comprising adding an amount of a second reagent to the solution, the second reagent having a pH different from the first reagent.

24. A method of protocol monitoring, the method comprising:
from each of a plurality of sequencing devices, receiving an indication of an amount of a reagent added to a solution to adjust the pH of the solution to within a range of a target pH;
determining an error state based at least in part on the received indications; and
notifying an administrator of the determined error state.

25. The method of claim 24, wherein determining the error state includes determining that an amount of adjustment is in excess.

26. The method of claim 24, wherein determining the error state includes determining a site level error.

27.-29. (canceled)
